(19) United States
(12) Patent Application Publication
(10) Pub. No.: US 2026/0224233 A1
(43) Pub. Date: Aug. 6, 2026

(54) DEVICES AND METHODS FOR FACILITATING GUIDEWIRE RE-ENTRY INTO A TRUE LUMEN

(71) Applicant: TruVue, Inc., Kalamazoo, MI (US)

(72) Inventor: Khaldoon ALASWAD, Grosse Point Park, MI (US)

(21) Appl. No.: 19/154,377

(22) PCT Filed: Feb. 6, 2024
(86) PCT No.: PCT/US2024/014681

§ 371 Date: Aug. 6, 2025

Related U.S. Application Data
(60) Provisional application No. 63/483,477, filed on Feb. 6, 2023.
(60) Provisional application No. 63/497,160, filed on Apr. 19, 2023.

(51) Int. Cl. A61B 17/22; A61B 8/12; A61B 34/20; A61M 25/01

(52) U.S. Cl. CPC A61B 17/22; A61B 8/12; A61B 34/20; A61B 2017/22038; A61B 2017/22095; A61B 2034/2063; A61M 25/0108

(57) ABSTRACT

Apparatuses and methods for facilitating reentry of a guidewire into a true lumen may include an elongate shaft having a proximal end and a distal end, and an intravascular ultrasound (IVUS) catheter engagement region (e.g., capsule) coupled to the distal end of the elongate shaft. An IVUS imaging window may be disposed in a sidewall of the IVUS engagement region. The IVUS engagement region may be configured to receive an IVUS catheter so that the IVUS catheter may image through the window to assist in imaging the vessel and guiding and/or confirming reentry of a guidewire into the true lumen of the vessel.

### CLAIM OF PRIORITY

This patent application claims priority to U.S. provisional patent application No. 63/483,477, titled “DEVICES AND METHODS FOR FACILITATING GUIDEWIRE RE-ENTRY INTO A TRUE LUMEN,” filed on Feb. 6, 2023, and U.S. provisional patent application No. 63/497,160, titled “DEVICES AND METHODS FOR FACILITATING GUIDEWIRE RE-ENTRY INTO A TRUE LUMEN,” filed on Apr. 19, 2023, each of which is herein incorporated by reference in its entirety.

### BACKGROUND

There are thousands of percutaneous coronary interventions (PCI) performed every year in the United States to treat chronic total occlusions (CTO) of coronary vessels, and even more worldwide. A greater number of CTO procedures are performed in the peripheral vessels. In coronary procedures, a guidewire may be used to cross an obstruction within the vessel, and the guidewire may then serve as a rail over which other diagnostic or therapeutic devices may be delivered to the treatment site. However, a large number of such procedures fail, often due to the nature of the occlusion and related problems with controlling the guidewire. For example, inadvertent entry of the guidewire into the surrounding layers of a blood vessel wall may require a device to assist in reentry of the wire back into the vessel true lumen. Alternative treatments can be challenging to learn or perform. Therefore, improvement devices and methods for treating occlusions are desirable.

### SUMMARY OF THE DISCLOSURE

Described herein are methods and apparatuses, including devices and systems (e.g., reentry ultrasound engagement catheters), that may facilitate the reentry of a guidewire into the true lumen of a vessel. These apparatuses and methods are generally configured to interact with one or more guidewires and an intravascular ultrasound (IVUS) apparatus. Referred to as reentry ultrasound engagement catheters, these apparatuses may include an elongated body, e.g., a microcatheter body, which houses a first lumen for guiding a reentry guidewire. They may also incorporate an IVUS engagement region, which may be configured as a capsule or an elongated body, that includes an ultrasound window, allowing proper orientation of the IVUS device relative to the rest of the apparatus and in particular the reentry guidewire exit port. This may enable visualization of the distal end of the elongated microcatheter body, especially the distal end of the first lumen and thus the reentry guidewire, through the ultrasound window. Furthermore, the IVUS engagement region may be designed to accommodate or interact with a second guidewire, such as a subintimal wire (or IVUS guidewire). The IVUS engagement region may feature a distal opening for passing the second guidewire and may incorporate a channel or a rapid exchange region for engaging the second guidewire and/or the IVUS device.

The methods described here may be employed as part of, or alongside, an antegrade, dissection, and reentry (ADR) procedure. In such a procedure aimed at crossing a stenotic lesion, the process typically involves creating a dissection, advancing a wire subintimally across the lesion within the vessel, and then reintroducing the wire, typically a guidewire, into the vessel's true lumen from the subintimal space. This maneuver establishes a pathway through which a diagnostic or therapeutic catheter can be passed to traverse the occlusion, allowing for further evaluation or treatment of the occlusion. As previously mentioned, ultrasound, including IVUS, can assist and guide this process. However, utilizing IVUS can be challenging, particularly when navigating and controlling both the guidewire and the IVUS device simultaneously. Therefore, the methods and apparatuses described herein offer a potentially simpler and more effective solution.

For example, described herein are systems for facilitating reentry of a guidewire into a true lumen that include: an elongate shaft having a proximal end and a distal end and a reentry guidewire lumen extending therethrough, the reentry guidewire lumen having an exit port at a distal end region of the elongate shaft; an intravascular ultrasound catheter (IVUS) engagement region, configured as a capsule, coupled to the distal end of the elongate shaft; and an IVUS imaging window disposed in a sidewall of the capsule, wherein the capsule is configured to receive an intravascular ultrasound (IVUS) catheter an to orient the IVUS catheter to image through the IVUS imaging window.

Any of these system may include one or more guides in (and/or adjacent to) the exit port to deflect a reentry guidewire laterally relative to a long axis of the elongate shaft. The guide may comprise: a ramp, a channel, or a deflector. Any of these systems may optionally include the guidewires (e.g., reentry guidewire) and/or the IVUS catheter.

The IVUS imaging window may be positioned laterally adjacent to the exit port. In some examples the IVUS imaging window comprises a cut-out region.

The capsule may comprise a porous material. In some example the capsule comprises a distal opening for an IVUS guidewire.

Any of these apparatuses may include one or more ultrasound markers on the distal end region of the elongate shaft and/or on the capsule. Alternatively or additionally, any of these apparatuses may include one or more engagement features within the capsule configured to orient the IVUS catheter. In some examples these apparatuses may include one or more attachments for releasably securing an elongate body of the IVUS catheter to the elongate body.

Also described herein are methods for using any of these apparatuses to guide reentry of a guidewire into the true lumen. For example, a method for facilitating reentry of a guidewire into a true lumen of a vessel, the method comprising: advancing an elongate shaft with a capsule toward an occlusion in the vessel; advancing the capsule through a subintimal space of the vessel and positioning the capsule distal of the occlusion; disposing an intravascular ultrasound (IVUS) catheter in the capsule; re-entering a guidewire from the subintimal space into a true lumen of the vessel while imaging the guidewire with the IVUS catheter to ensure the guidewire is positioned and advanced correctly.

In some examples the IVUS engagement region may be an elongate region. For example, a system for facilitating reentry of a guidewire into a true lumen may include an elongate shaft having a proximal end and a distal end; a reentry guidewire lumen extending between the proximal end and a distal end, the reentry guidewire lumen configured to slidably receive a reentry guidewire; an exit port fluidly connected to the reentry guidewire lumen at a distal end region of the elongate shaft; an intravascular ultrasound (IVUS) lumen extending between the proximal end and the distal end of the elongate shaft, the IVUS lumen configured to slidably receive an IVUS catheter; and an IVUS imaging window disposed in a sidewall of the elongate shaft in communication with the IVUS lumen and distal or adjacent to the exit port.

Any of these systems may include a guides in or adjacent to the exit port to deflect a reentry guidewire laterally relative to a long axis of the elongate shaft. As mentioned, the guide may comprise: a ramp, a channel, or a deflector.

The IVUS imaging window may be positioned laterally adjacent to the exit port. In some examples the IVUS imaging window comprises a cut-out region. Any of these apparatuses may include one or more ultrasound markers on the distal end region of the elongate shaft. The apparatus may include one or more engagement features within the IVUS lumen configured to orient the IVUS catheter.

As mentioned, also described are method for facilitating reentry of a guidewire into a true lumen of a vessel including methods using a system as described above. For example, a method may include: advancing an elongate shaft having a first lumen and a second lumen toward an occlusion in the vessel; advancing the elongate shaft over a guidewire disposed the first lumen through a subintimal space of the vessel; advancing an intravascular ultrasound catheter (IVUS) through the second lumen; and advancing a reentry guidewire through the first lumen and re-entering the reentry wire from the subintimal space into the true lumen of the vessel while imaging the reentry guidewire with the IVUS catheter to ensure that the reentry guidewire is positioned and advanced correctly.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A and 1B show an example of a reentry ultrasound engagement catheter that works with a separate IVUS catheter to facilitate reentry of a wire into a vessel true lumen or other target location. FIG. 1A shows the reentry ultrasound engagement catheter engaged with the IVUS device (IVUS catheter). FIG. 1B shows the reentry ultrasound engagement catheter.

FIG. 2 shows an example of a guidewire being advanced across a total occlusion via the subintimal space.

FIGS. 3A-3B show another example of a reentry ultrasound engagement catheter apparatus similar to that shown in FIGS. 1A-1B. FIG. 3A shows the reentry ultrasound engagement catheter engaged with an IVUS catheter and reentry wire. FIG. 3B shows the reentry ultrasound engagement catheter engaged with the reentry wire (without the IVUS catheter).

FIG. 4 shows the reentry ultrasound engagement catheter of FIG. 1 with an IVUS catheter in the subintimal space.

FIG. 5 shows an example of a reentry wire re-entering the vessel true lumen from the subintimal space.

FIG. 6 shows an example of a wire traversing the occlusion.

FIGS. 7A-7B illustrate examples of IVUS engagement region that may be included as part of the apparatuses described herein. FIG. 7A shows a first example of an apparatus including an IVUS engagement region configured as a capsule. FIG. 7B shows a second example of an apparatus including an IVUS engagement region configured as a capsule.

FIGS. 8A-8B shows an example of a reentry ultrasound engagement catheter apparatus that is configured to work with a separate IVUS catheter to facilitate reentry of a wire into a vessel true lumen or other target location.

FIGS. 9A-9B shows an example of a reentry ultrasound engagement catheter apparatus that works with a separate IVUS catheter to facilitate reentry of a wire into a vessel true lumen or other target location.

### DETAILED DESCRIPTION

Described herein are a methods and apparatuses (e.g., devices, systems, etc., including reentry ultrasound engagement catheters) for facilitating reentry of a guidewire into the true lumen of a vessel. In general, these apparatuses and methods are configured to engage with one or more guidewires and an intravascular ultrasound, e.g., IVUS, apparatus. These apparatuses may be referred to as reentry ultrasound engagement catheters. In some examples these apparatuses include an elongate body (e.g., a microcatheter body) comprising a first lumen for guiding a reentry guidewire, and an IVUS engagement region, configured as a capsule or an elongate body with an ultrasound window configured to orient the IVUS device so that the distal end of the elongate microcatheter body, and particularly the distal end of the first lumen (and therefore the reentry guidewire) may be visualized through the ultrasound widow. The IVUS engagement region may also be configured to pass or engage a second guidewire (e.g., a subintimal wire). In some case the IVUS engagement region may include a distal opening for passing the second guidewire. In some examples the IVUS engagement region may include a channel and/or a rapid exchange region for engaging the second guidewire and/or the IVUS device.

The methods described herein may be used as part of, or in conjunction with an antegrade, dissection, and reentry (ADR) procedure. An ADR procedure for crossing a stenotic lesion may include creating a dissection, and advancing a wire subintimally across a lesion (e.g., occlusion, clot, etc.) in the vessel, and then re-introducing the wire (e.g., guidewire) into vessel true lumen from the subintimal space. This may provide a pathway through which a diagnostic or therapeutic catheter may be passed in order to traverse the occlusion and allow further evaluation or treatment of the occlusion. As mentioned above, the process may be guided or assisted using ultrasound, including IVUS. Unfortunately, the use of IVUS may be challenging, particularly when navigating and controlling the guidewire as well as the IVUS device. Thus, the methods and apparatuses described herein may provide an easier and more effective solution.

In some examples the methods and apparatuses described herein may use a catheter, e.g., a reentry ultrasound engagement catheter, to help deliver a separate, discrete intravascular ultrasound catheter (IVUS) subintimally across an occlusion where the IVUS catheter can then be used to observe and help a physician guide a reentry wire from the subintimal space back into the vessel true lumen. The wire (reentry guidewire) can then be used as a rail over which other devices may be successfully delivered past the occlusion for further diagnosis and treatment (e.g., angioplasty catheters or stent delivery systems). This example is not intended to be limiting the examples disclosed herein may have other uses. For example, in addition to facilitating true lumen reentry from the subintimal space, the device described herein may also be used to facilitate entry of a wire into a side branch ostium from the true lumen of the vessel, or direct a wire to enter the subintimal space from the vessel lumen to create a dissection or allow drug delivery, or to puncture the vessel from the inside out toward a specific area, like a nearby vein or to create access to nearby healthy or unhealthy tissues like cancer. All of these procedures, which are not intended to be limiting, allow more accurate wire manipulation while under visualization of the IVUS catheter.

In general, intravascular ultrasound (IVUS) may be useful to guide the subintimal guidewire to re-enter the true lumen. The reentry ultrasound engagement catheters described herein may be configured to removably engage with and enhance control for orienting and positioning the IVUS catheters, particularly when transitioning between subintimal and true lumen of the vessel.

Thus, the reentry ultrasound engagement catheter apparatuses described herein may include an elongate body having or forming a lumen for a reentry guidewire and an IVUS engagement region that may extend distally from the exit port for the lumen of the reentry guidewire that is configured to engage with an IVUS catheter to allow and enhance the IVUS catheter to visualize the reentry guidewire as it extends from the exit port. The reentry guidewire may be directed out of the reentry ultrasound engagement catheter apparatus using one or more guides (e.g., ramps, channels, deflectors, etc.) of the reentry ultrasound engagement catheter apparatus and/or using imaging form the IVUS catheter held in position and/or orientation by the reentry ultrasound engagement catheter apparatus.

The elongate body of the reentry ultrasound engagement catheter apparatus may be configured as an elongate torquable body. The elongate torquable body may be configured as a microcatheter body. The body may be reinforced to improve torquability, e.g., by including a mesh or braid. In some cases the elongate microcatheter body may be a hypotube that cut (e.g., laser cut) to increase flexibility while maintaining torquability and/or column strength (pushability).

The reentry guidewire lumen may be configured to extend along the length of the elongate body of the reentry ultrasound engagement catheter, or it may be configured as a rapid-exchange configuration at the distal end region of the elongate body. The reentry guidewire lumen may include an exit port at the distal end region of the elongate body. The elongate body may be configured to steer, guide or direct the reentry guidewire off-axis relative to the elongate body so that when the distal end region of the elongate body is outside of the true lumen (e.g., the intima) of the vessel, the reentry guidewire may be directed laterally into the true lumen. Thus, in some examples the reentry guidewire lumen and/or exit port may be configured to steer, guide or direct the reentry guidewire into the true lumen, e.g., laterally off-axis. For example, the distal end of the elongate body (including within the reentry guidewire lumen and/or the exit port for the reentry guidewire lumen, and/or the region distal to the exit port) may include a guides (e.g., ramp, channel, deflector, etc., also referred to as “guide features”) that is configured to direct tip of the reentry guidewire laterally. In some examples the exit port for the reentry lumen may be lateral on the elongate body (e.g. on a side of the elongate body). In some cases the exit port is positioned relative to the IVUS engagement region so that the reentry guidewire may be visualized as the reentry guidewire exist the lumen, so that it may be steered and/or so that reentry may be confirmed.

In any of these apparatuses the IVUS engagement region may be coupled to and/or may be integral with, the elongate body. In some cases the IVUS engagement region is configured to extend distally of the elongate body, including distal to the reentry guidewire lumen exit port. In some examples the IVUS engagement region is configured as a distal capsule extending partially along the distal end of the apparatus. Alternatively, in some examples the IVUS engagement region is configured as an elongate body extending proximally and configured to contain much of the length of the IVUS catheter.

In general, the IVUS engagement region includes at least a region that is ultrasound transparent. For example, the IVUS engagement region may include an ultrasound (IVUS) window, e.g., and IVUS imaging window, through which ultrasound energy may be delivered to image the reentry of the guidewire and/or the vessel (e.g., occlusion). In some examples, the IVUS imaging window is configured to be at a distal end region of the IVUS engagement region; the IVUS imaging window may be positioned opposite to the reentry lumen exit port. The IVUS imaging window may be configured as a discrete region; the IVUS imaging window may be configured as an oval, rectangular, square, teardrop, triangular, circular, etc. shape. The shape may allow orientation of relative to the field of view. The IVUS imaging window may direct the user's attention. In some examples the non-window region of the IVUS engagement region may be configured to reduce the intensity of (e.g., to partially or fully occlude) the ultrasound imaging by the IVUS catheter. In some cases the IVUS imaging window may be a cut-out region or opening through a lateral region (e.g., side) of the IVUS engagement region. In some cases the IVUS imaging window may be formed of a material that transparent to ultrasound at the wavelengths used for imaging. Optionally, in some case the entire (or most of) the IVUS engagement region may be formed of an ultrasound transparent material. The IVUS engagement region may be configured to include one more additional or alternative ultrasound-dense markers (fiduciary markers). Optionally in some examples the apparatus may include one or more markers (IVUS imaging markers), including at positions that are opposite from the reentry catheter exit port and/or adjacent to the exit port.

In variations including an IVUS imaging window, the window may generally be positioned to allow imaging of the reentry wire as it leaves the reentry guidewire lumen out of the elongate body (e.g., microcatheter). In some examples the IVUS engagement region may extend distal to a distal end of the elongate body.

In any of the IVUS engagement regions described herein the engagement region may generally be configured to releasably secure the IVUS catheter distal end region and/or to orient the IVUS catheter so that it may image the occlusion and/or reentry wire. For example, the IVUS engagement region may be configured to hold and/or orient the imaging portion of the IVUS catheter so that it may image the vessel relatively un-occluded (including by the reentry ultrasound engagement catheter) and/or may image the reentry guidewire to confirm and/or assist in reentry into the true lumen.

In some cases the IVUS engagement region may include one or more engagement features. The engagement features may hold, position and/or orient the IVUS catheter. For example, the distal IVUS engagement region may be configured to engage with the distal end region of the IVUS catheter by including one or more notches, tapered regions, etc. that may position and/or orient the IVUS catheter imaging component(s). In some cases the IVUS catheter may be configured to include a distal channel and/or opening (exit port) for an IVUS catheter guidewire (e.g., a “guidewire window”). Thus, in general the IVUS engagement region may be configured to be used with an IVUS catheter guidewire.

The IVUS engagement region may be formed of any appropriate material. For example, the IVUS engagement region may be formed of a mesh material. In some cases the IVUS engagement region may be formed as a cylinder or tapered cylinder, cone, etc. The IVUS engagement region may be porous or closed along the sides. The IVUS engagement region may include a proximal opening for engaging with/inserting the IVUS catheter and/or a guidewire that may be used with the IVUS catheter. In any of these apparatuses the inner diameter of the IVUS engagement region may be configured to hold the IVUS catheter (e.g., the IVUS engagement region may have an inner diameter that is larger than a maximum outer diameter of the IVUS device).

In some cases the elongate body of the reentry ultrasound engagement catheter and the IVUS engagement region are formed as an integral piece. For example, the elongate body of the reentry ultrasound engagement catheter may be integral with the IVUS engagement region. Alternatively, the elongate body and the IVUS engagement region may be adjacent to each other, but coupled together at the distal end region. In some cases, the reentry ultrasound engagement catheter is configured to receive a rotating IVUS device. In some cases the IVUS engagement region may be tandem to the elongate body forming the lumen for reentry wire.

In variations in which the IVUS engagement region is coupled to the elongate body at the distal end region and does not extend fully proximally, the apparatus may include one or more IVUS catheter engagement connectors along the length of the elongate body. For example, the elongate body may include one or more attachments for securing (releasably securing) the body of the IVUS catheter. The engagement connectors may be configured as one or more of: snaps, clips, loops, etc. In general, the engagement connector may be positioned along the length or just distal end region of the elongate body.

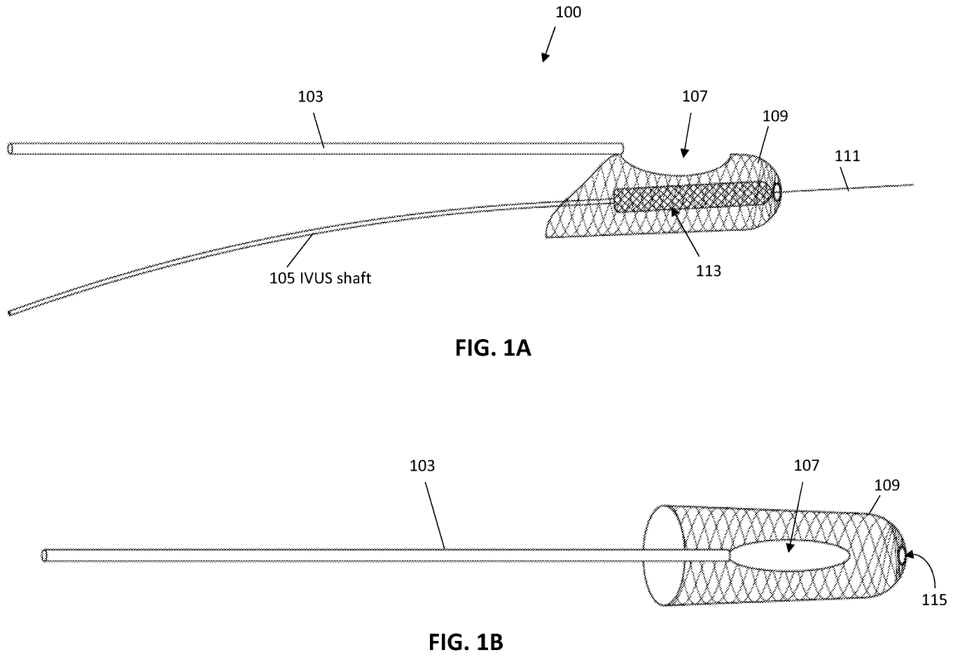

FIGS. 1A and 1B illustrate one example of an apparatus (e.g., a reentry ultrasound engagement catheter apparatus) that is configured as a device that may be used in conjunction with a separate IVUS catheter to facilitate reentry of a wire into a true lumen of the vessel. In some examples the reentry ultrasound engagement catheter device itself does not include an ultrasound transducer; instead the IVUS transducer is provided by a separate IVUS catheter. Alternatively in some examples the reentry ultrasound engagement catheter may include an integrated IVUS catheter.

In examples using an existing IVUS catheter (such as a phased array IVUS catheter or a rotational IVUS catheter) the reentry ultrasound engagement catheter may be configured to provide a more cost effective than using a reentry device that has an integrated ultrasound imaging transducer and then disposing of the device after single use.

The reentry ultrasound engagement catheter 100 in FIGS. 1A and 1B includes an elongate body (e.g., shaft) 103 with an IVUS catheter engagement 109 configured as a capsule at the distal end of the shaft. The shaft may be a microcatheter and may be a single or multi-lumen catheter and the shaft may be formed from polymers, metals, or other material known in the art. The shaft may be braided or have any desired stiffness in order to give it the desired mechanical properties (e.g., stiffness, column strength, torqueability, etc.). The elongate shaft may have a lumen for a reentry wire (“reentry guidewire lumen”) and that lumen may extend along the length of the elongate body, or may be a rapid exchange lumen or an over the wire lumen. In some cases where the lumen is a rapid exchange lumen, the distal exit port may be disposed at the distal-most end of the shaft, and the proximal port may be disposed closer to the distal port than the proximal-most end of the elongate shaft. In some examples the lumen is an over the wire lumen, and the distal port may be disposed at the distal-most end of the shaft (or optionally a lateral side thereof) and the proximal port may be disposed at the proximal-most end of the shaft. The wire lumen may be used to introduce a reentry wire as will be discussed in greater detail below. The proximal end of the shaft may include a hub with Luer connectors for facilitating connections with other components such as tubing, syringes, etc. The shaft may have additional lumens for other guidewires, fluid infusion or removal, etc. as needed.

The distal capsule 109 may be coupled to the distal end of the shaft 103 and the shaft may include a reentry guidewire lumen, as mentioned. The distal end of the reentry guidewire lumen may be disposed adjacent to the center portion and proximal edge of an IVUS window 107 on the capsule. A reentry wire may be advanced via the reentry guidewire lumen to exit in view of the IVUS imaging (e.g., IVUS beam(s)) and then be directed toward the area of interest, including the true lumen, a side branch, or the vessel wall. The distal capsule 109 may include a distal aperture or port 115 where a guidewire from the IVUS catheter may exit the capsule. In any of these examples the IVUS catheter engagement region (e.g., capsule) may be a radially expandable/collapsible cylindrical or conical structure with a central chamber configured to receive and house an IVUS catheter. During delivery, the capsule may be in a collapsed configuration, and once delivered to a desired location, the capsule may be expanded into the expanded configuration. In other examples, the capsule may remain in the expanded configuration delivery. The capsule may receive and carry an IVUS catheter during advancement through a vessel to the desired treatment area, or the IVUS catheter may be advanced separately over a guidewire into the capsule once the capsule has been delivered to the desired treatment area. In FIG. 1A the reentry ultrasound engagement catheter 100 is shown engaged with an ultrasound catheter including an IVUS imaging array 113 at a distal end of an IVUS catheter shaft 105; an IVUS guidewire 111 is shown through the IVUS catheter.

The IVUS catheter engagement region 109 has an IVUS window which may be a cutout region in the wall of the capsule which allows the IVUS catheter to image in a sideways or lateral direction through the wall of the capsule relative to the longitudinal axis of the catheter. The IVUS catheter may be any IVUS catheter such as those commercially available (e.g. phased array or rotational IVUS catheters) and used by practitioners. The IVUS catheter may have a guidewire lumen that receives a guidewire so that the IVUS catheter may be advanced over the guidewire to the target treatment area and into the capsule, or if the IVUS catheter is disposed in the capsule during delivery, the two may be advanced together toward the treatment area over a guidewire or without a guidewire. Once delivered, the ultrasound transducer on the IVUS catheter may be rotated in order to align the transducer on IVUS catheter with the IVUS window in the capsule to permit imaging through the IVUS window

FIGS. 7A-7B show several examples of IVUS catheter engagement regions, configured as capsules 709, 709′. In any of the examples of devices disclosed herein, the IVUS catheter engagement region (sometimes also referred to as capsules or as an IVUS sleeve) can be open at the distal end or the IVUS catheter engagement region may have a closed distal end that includes a wire opening at the distal tip. Both of the capsules shown include an open distal end 715 that may pass a guidewire (e.g., an IVUS guidewire). The shaft 705 coupled to the IVUS catheter engagement region can be rotated to face the true lumen or any area of interest in the vessel, like the ostium of a side branch. The shaft 705 of the elongate body of the apparatus may include radiopaque markers or echogenic markers at various locations in order to allow the operator to visualize the location of various device features during use. For example, a marker may be disposed at the distal end of the elongate shaft, or adjacent the proximal end and/or distal end of the IVUS window, adjacent the proximal and/or distal ends of the capsule, or adjacent any of the lumen ports, etc.

In examples where the IVUS visual field is 360° circumferentially around the capsule, the IVUS window 707 will facilitate visualization in one direction while the other directions may be blocked by the reentry device. The shaft with capsule with or without simultaneous delivery of the IVUS catheter may be advanced to the stenotic lesion and further advanced past the occlusion using the antegrade, dissection and reentry (ADR) procedure discussed above. The IVUS catheter can then be used to image and ensure that the reentry wire passes from the subintimal space into the true lumen properly, or helps the operator know if repositioning is needed. Other uses of the device are disclosed above.

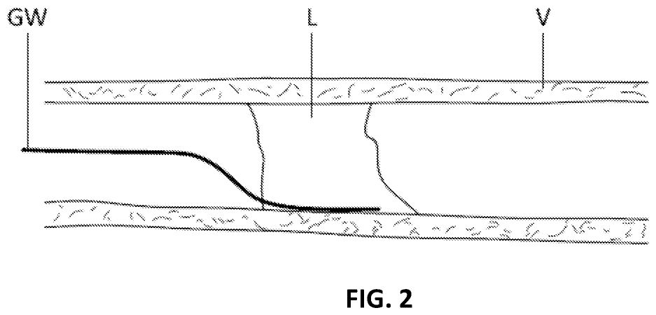

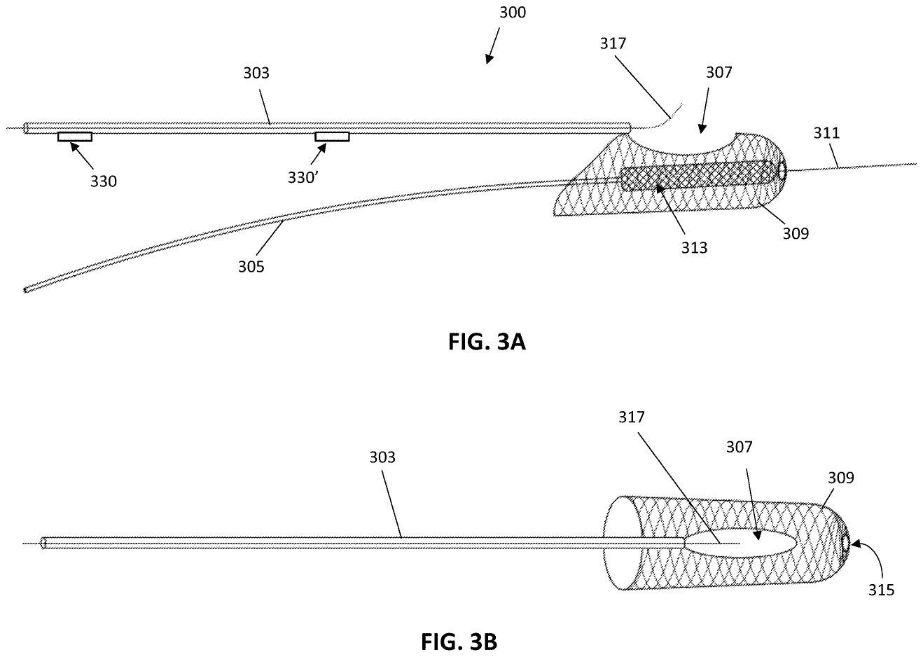

FIG. 2 shows a guidewire GW advanced distally through a vessel V where a dissection is created so that the wire can be advanced subintimally across the lesion L and past the occlusion but without reentry into the true lumen. FIGS. 3A-3B show that after the guidewire has been positioned, the reentry ultrasound engagement catheter device described above may be advanced over the guidewire subintimally. Optionally, an IVUS catheter (IVUS) may be delivered simultaneously with the reentry ultrasound engagement catheter subintimally, or in another example, the IVUS catheter may be delivered and received into the reentry ultrasound engagement catheter after the reentry ultrasound engagement catheter has been delivered to the desired location. FIG. 3A is shown without the vessel surrounding the reentry ultrasound engagement catheter and IVUS catheter for ease of visualization.

In FIG. 3A the reentry ultrasound engagement catheter 300 is similar to that shown in FIG. 1A and includes and elongate body 303 with a reentry guidewire lumen (out of which a reentry guidewire 317 extends) and an IVUS engagement region 309 configured as a tapered capsule including a distal opening 315 for the IVUS guidewire 311. The IVUS catheter includes a shaft 305 and a distal IVUS sensor 313 (e.g., transducer array).

In FIG. 3 A the elongate body 303 of the reentry ultrasound engagement catheter device 300 also includes a plurality (e.g., two are shown) of securements 330, 330′ for coupling the IVUS shaft 305 to the elongate body (not shown). The securement may radially secure the IVUS shaft, while allowing it to slide axially, so that it may navigate turns without inhibiting bending of the elongate body of the reentry ultrasound engagement catheter. The capsule 309 includes an IVUS imaging window 307.

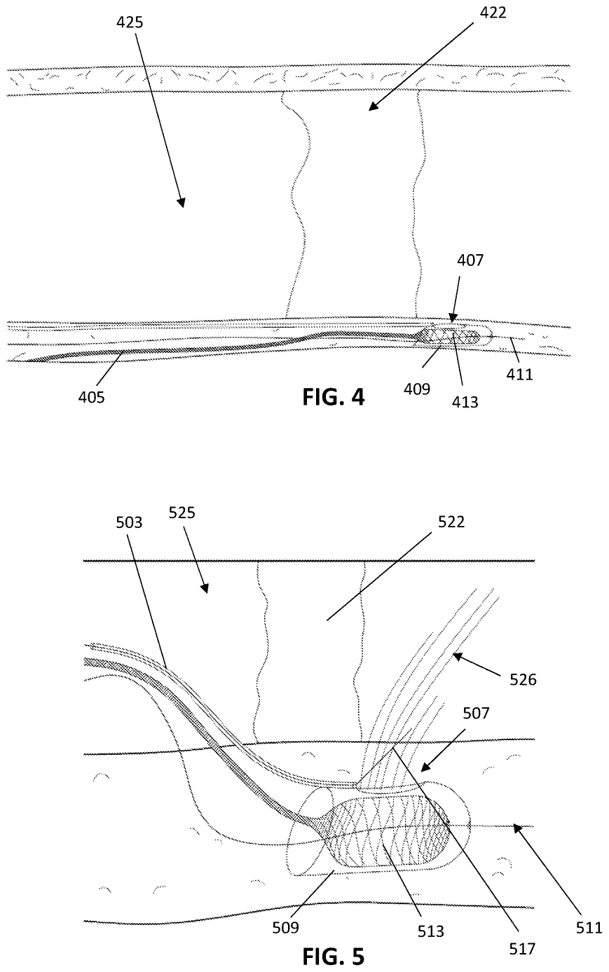

FIG. 4 shows the reentry ultrasound engagement catheter and IVUS catheter disposed in the subintimal space. Once the reentry ultrasound engagement catheter and the IVUS catheter are properly positioned, e.g., with the IVUS catheter 413 within the capsule 409 of the reentry ultrasound engagement catheter the IVUS catheter may be rotated, e.g., by rotating the IVUS catheter shaft 405, in order to align the ultrasound imaging transducer on the IVUS catheter with the imaging window 407 in the reentry ultrasound engagement catheter and a reentry wire may be advanced through the capsule shaft lumen, and advanced distally so that it exits the lumen at the distal end of the shaft and can then re-enter the vessel true lumen or be directed to any other desired location such as a side branch or vessel wall.

The wire may exit from the elongated shaft distal port at an angle from 0-135° and the IVUS catheter may be oriented to facilitate wire visualization by the ultrasound beam. In FIG. 4 the lumen 425 of the vessel is occluded by an occlusion 422. A separate guidewire (e.g., an IVUS guidewire 411) may be used to position the IVUS catheter, including within the reentry ultrasound engagement catheter.

FIG. 5 show the reentry being performed while the IVUS catheter 513 images 526 the advancement of the reentry guidewire 517 to ensure proper positioning and advancement. The IVUS catheter helps to guide the wire puncturing from the subintimal space back into and entering the true lumen 525 to help prevent the wire from exiting the vessel and/or expanding the dissection. In FIG. 5, the reentry ultrasound engagement catheter includes an elongate body (e.g., shaft) 503 with an IVUS engagement region (e.g., capsule) 509 coupled to the distal end region, including an IVUS imaging window 507 out of which the IVUS device 513 may image. The IVUS catheter may be guided in part by an IVUS guidewire 511 and may be positioned on the opposite side of the occlusion 522 of the lumen; the lumen and occlusion may be imaged using the IVUS catheter.

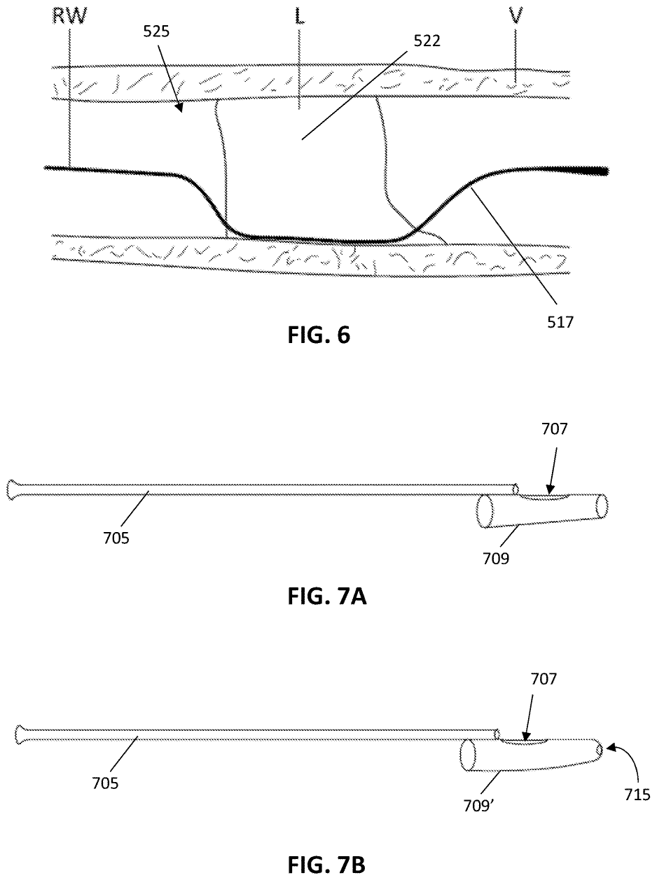

FIG. 6 shows the capsule catheter removed along with the other devices, leaving only the reentry wire RW 517 in the vessel V, providing a path across the lesion L (e.g., occlusion 522) within the vessel lumen 525, so that other devices can use the reentry wire as a rail to guide them across the lesion. The reentry wire may be referred to as a guidewire or simply a wire, equivalently. In some examples, the reentry wire may be replaced with another more suitable wire using wire exchange techniques known in the art (e.g., using a microcatheter). Angioplasty or stenting or other diagnostic or therapeutic devices may be advanced over the wire and procedures may then be performed to remove or otherwise treat the occlusion. Of course, one of skill in the art will appreciate that the method described above may be modified in a number of ways and therefore is not intended to be limiting. Various steps may be replaced with other steps, and the order to steps may be modified as desired by the operator.

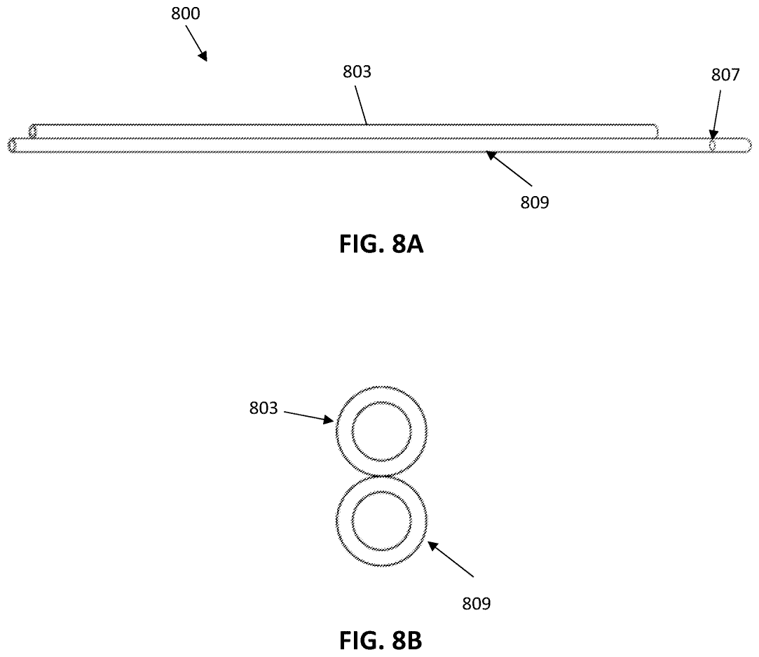

FIGS. 8A-8B shows another example of a reentry ultrasound engagement catheter 800 that may be used to facilitate the use of a separate, discrete IVUS catheter during a reentry procedure or other use described herein. In this example the IVUS engagement region 809 is coupled to and extends along the length of the elongate body 803, which may otherwise be similar or identical to the elongate bodies described above. In FIG. 8A, the IVUS engagement region include an elongate lumen having a distal IVUS window region 807, and the IVUS catheter may be inserted through the IVUS engagement region 809 from the proximal end, either directly or over an IVUS guidewire (not shown). FIG. 8B shows a section through the device of FIG. 8A.

The reentry ultrasound engagement catheter 800 in FIG. 8A includes a first elongate body (shaft) 803 and the IVUS engagement region 809 is configured as a second elongate shaft. The two elongate shafts may be coupled together such that a cross-section taken orthogonally relative to the longitudinal axis of the catheter may be a figure eight shaped geometry, as seen in FIG. 8B. The shafts may be co-extruded to form an integral body, or the two shafts may be bonded together, or other techniques known in the art may be used to couple the two shafts together. Either shaft or both may be braided in order to provide torqueability. The first elongate shaft has a proximal end and a distal end. The proximal end may include a hub, such as Luer connector so that the proximal end may be fixedly or releasably coupled with other devices such as fluid tubing, a syringe or other equipment. The distal end may have an exit port. The first elongate shaft may have one or more lumens extending between the proximal and distal ends. At least one lumen may be sized to receive a reentry wire that can be observed and guided by an IVUS catheter disposed in the second elongate shaft which has a proximal end and a distal end, and at least one lumen extending therebetween to slidably receive the IVUS catheter. The second elongate shaft may also have a hub such as a Luer connector to allow the second elongate shaft to be coupled to other devices such as tubing, syringes, hemostasis valves, etc. In use, the device of FIG. 8 may be advanced over a wire which has already been positioned in the subintimal space. The device is slidably advanced over the wire which is disposed in the lumen of the first elongate shaft. The lumen in the first elongate shaft may be configured as an over the wire lumen where the proximal port is at the proximal end of the first elongate shaft and the distal port is disposed at the distal end of the first elongate shaft. Alternatively, the lumen in the first shaft may be configured as a monorail (rapid exchange) lumen where the distal-most wire exit port on the first elongate shaft is at the distal-most end of the first elongate shaft and the proximal wire port is located at a proximal port on the second elongate shaft that is disposed closer to the distal end of the first elongate shaft than the proximal end of the first elongate shaft.

In general, the IVUS catheter may be a phased array IVUS catheter or a rotational IVUS catheter. The IVUS catheter may be pre-loaded into the lumen of the second elongate shaft as the reentry ultrasound engagement catheter device is advanced toward the occlusion and subintimally across the occlusion, or the IVUS catheter may be slidably advanced through the second elongate shaft after the reentry ultrasound engagement catheter device has been positioned subintimally. The IVUS lumen in the second elongate shaft may be configured as an over the wire lumen as previously described above, or the IVUS lumen may be configured as a rapid exchange lumen as previously described above. Once the reentry ultrasound engagement catheter device and the IVUS catheter have been deployed in a desired location, the reentry ultrasound engagement catheter device is used similar to other examples disclosed herein. A reentry wire is advanced through the lumen of the first elongate shaft and then it is observed and guided to re-enter the true lumen using the IVUS catheter disposed in the second elongate shaft. Once the reentry wire has re-entered the true lumen, the reentry ultrasound engagement catheter device and IVUS catheter may be retracted leaving on the reentry wire and then further diagnostic of therapeutic devices can be advanced across the occlusion using the reentry wire or another wire that has replaced the reentry wire.

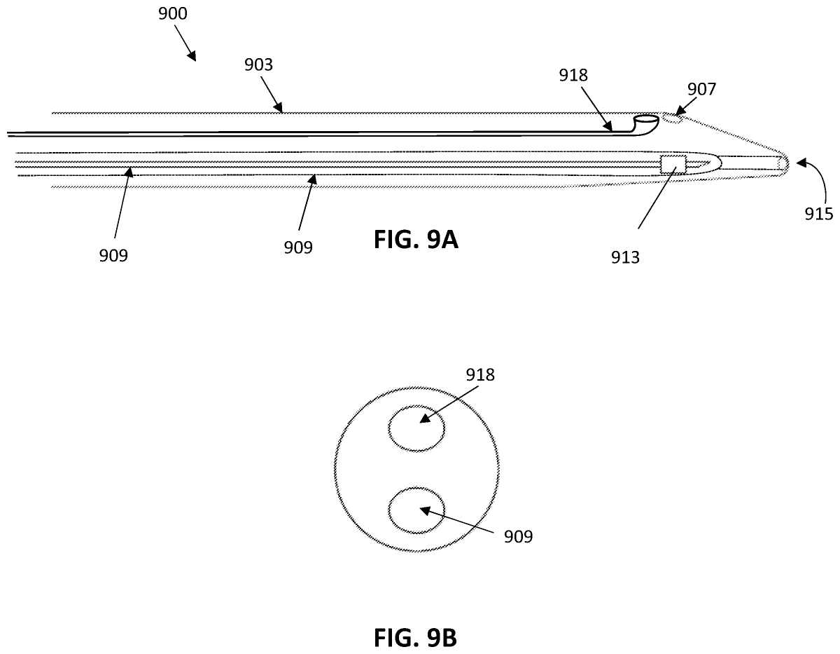

FIGS. 9A-9B shows another example of a reentry ultrasound engagement catheter 900 that may be used to facilitate the use of a separate, discrete IVUS catheter during a reentry procedure or other use described herein. In this example, the reentry guidewire lumen 918 is integrated into the same elongate body as the IVUS engagement region 909. The IVUS engagement region is formed as a second lumen extending parallel to the reentry guidewire lumen, and extends distally further than the reentry guidewire lumen and exit port. The IVUS catheter may be inserted into the IVUS engagement region and may, in some examples, rotate within the IVUS engagement region. In FIG. 9A the apparatus may also include an IVUS imaging window 907 that may be distal to (and/or in some examples may overlap with) the exit port of the reentry guidewire. This window may be open or may be formed of an ultrasound transparent material, as described above.

Thus, the reentry ultrasound engagement catheter device 900 in FIG. 9A includes an elongate body (e.g., shaft) 903 with a proximal and distal end. The elongate shaft has at least two lumens extending between the proximal and distal ends. As mentioned, the first lumen is for the reentry guidewire and the second lumen 909 is configured as the IVUS engagement region, for the separate, discrete IVUS catheter 913. The proximal end of the catheter (not shown) may include one or more hubs, such as Luer connectors to allow each lumen to be coupled to other devices such as tubing, syringes, hemostasis valves, etc. The first lumen may have either an over the wire configuration (OTW) or a monorail (rapid exchange, RX) configuration. In the OTW configuration the lumen has a proximal port which is at the proximal end of the catheter and the distal exit port is at the distal end of the catheter. In the RX configuration, the lumen has a distal port which is at the distal end of the catheter and the proximal port is closer to the distal end of the catheter than the proximal end of the catheter. Therefore, there may be several distal exit ports, including a distal exit port at the distal end of the catheter, or a distal port axially proximal from the distal end of the catheter for the first lumen when in the RX configuration. There also may be an exit port for the reentry wire that is adjacent the RX distal port. In some examples the reentry wire port and the RX port may be the same port. The IVUS engagement region may include a distal opening 915 for the IVUS guidewire to exit.

The second lumen (e.g., the IVUS engagement region) may be sized and configured to slidably receive a separate and discrete IVUS catheter such as a rotational IVUS catheter, or a phased array IVUS catheter. The proximal port is at the proximal end of the catheter and the distal port may be the same distal port as the first lumen distal port in the OTW configuration, although in alternative examples, the second lumen may be configured as a rapid exchange lumen.

The cross-section of the catheter may be as shown in FIG. 9B, where the catheter is an outer circular shape with the two circular lumens in the catheter wall. Catheter cross-section and lumen cross-section geometry may be circular, oval, elliptical, square, rectangular, or any other desired shape.

In use, the device of FIGS. 9A-9B is advanced over a guidewire slidably disposed in the first lumen that has already been placed subintimally to cross the occlusion. In the RX configuration, the wire enters the catheter from the distal-most port and then exits the RX proximal port. The IVUS catheter, such as a rotational IVUS catheter, may be pre-loaded into the second lumen as the catheter is advanced over the wire subintimally, or the IVUS catheter may be inserted into the second lumen after the catheter has been properly subintimally positioned. The IVUS catheter may then be used to visualize and guide a reentry wire that is placed in the first lumen to re-enter the true lumen. The reentry wire may exit a reentry port that is separate from the RX port, or the reentry wire port may be the same as the RX port. Once the reentry wire has been properly advanced into the true lumen, the catheter may be retracted, and other therapeutic or diagnostic devices may be advanced over the reentry wire across the lumen.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document. In addition, as described, these figures may illustrate a catheter that may be used in combination with a separate intravascular ultrasound (IVUS) catheter to facilitate wire reentry from a subintimal space into a vessel true lumen during treatment of an obstruction. The devices and methods disclosed herein may also be used to help direct a guidewire to a specific area, side branch, or lumen using an IVUS catheter.

### EXAMPLES

The following, non-limiting examples, detail certain aspects of the present subject matter to solve the challenges and provide the benefits discussed herein, among others.

Example 1 is a system for facilitating reentry of a guidewire into a true lumen, comprising an elongate shaft having a proximal end and a distal end; a capsule coupled to the distal end of the elongate shaft, and a window disposed in a sidewall of the capsule, wherein the capsule is configured to receive an intravascular ultrasound catheter (IVUS), and wherein the IVUS catheter is configured to image through the window.

Example 2 is the system of Example 1, further comprising the IVUS catheter.

Example 3 is a method for facilitating reentry of a guidewire into a true lumen of a vessel, comprising: advancing an elongate shaft with a capsule toward an occlusion in the vessel; advancing the capsule through a subintimal space of the vessel and positioning the capsule distal of the occlusion; disposing an intravascular ultrasound catheter (IVUS) in the capsule; and re-entering a guidewire from the subintimal space into a true lumen of the vessel while imaging the guidewire with the IVUS catheter to ensure the guidewire is positioned and advanced correctly. Example 4 is a system for facilitating reentry of a guidewire into a true lumen, comprising: an elongate shaft having a proximal end and a distal end; a first lumen extending between the proximal end and a distal end, the first lumen configured to slidably receive a reentry wire; and a second lumen extending between the proximal end and the distal end, the second lumen configured to slidably receive an IVUS catheter.

Example 5 is a method for facilitating reentry of a guidewire into a true lumen of a vessel, comprising: advancing an elongate shaft having a first lumen and a second lumen toward an occlusion in the vessel; advancing the elongate shaft over a guidewire disposed the first lumen through a subintimal space of the vessel; advancing an intravascular ultrasound catheter (IVUS) through the second lumen; and advancing a reentry wire through the first lumen and re-entering the reentry wire from the subintimal space into the true lumen of the vessel while imaging the reentry wire with the IVUS catheter to ensure that the reentry wire is positioned and advanced correctly.

In Example 6 the apparatuses or method of any one or any combination of Examples 1-5 can optionally be configured such that all elements or options recited are available to use or select from.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for facilitating reentry of a guidewire into a true lumen, the system comprising:
an elongate shaft having a proximal end and a distal end and a reentry guidewire lumen extending therethrough, the reentry guidewire lumen having an exit port at a distal end region of the elongate shaft;
an intravascular ultrasound catheter (IVUS) engagement region, configured as a capsule, coupled to the distal end of the elongate shaft; and
an IVUS imaging window disposed in a sidewall of the capsule, wherein the capsule is configured to receive an intravascular ultrasound (IVUS) catheter to orient the IVUS catheter to image through the IVUS imaging window.

2. The system of claim 1, further comprising a guides in or adjacent to the exit port to deflect a reentry guidewire laterally relative to a long axis of the elongate shaft.

3. The system of claim 2, wherein the guide comprises: a ramp, a channel, or a deflector.

4. The system of claim 1, further comprising the reentry guidewire.

5. The system of claim 1, wherein the IVUS imaging window is positioned laterally adjacent to the exit port.

6. The system of claim 1, wherein the IVUS imaging window comprises a cut-out region.

7. The system of claim 1, wherein the capsule comprises a porous material.

8. The system of claim 1, wherein the capsule comprises a distal opening for an IVUS guidewire.

9. The system of claim 1, further comprising one or more ultrasound markers on the distal end region of the elongate shaft and/or on the capsule.

10. The system of claim 1, further comprising one or more engagement features within the capsule configured to orient the IVUS catheter.

11. The system of claim 1, further comprising the IVUS catheter.

12. The system of claim 1, further comprising one or more attachments for releasably securing an elongate body of the IVUS catheter to the elongate body.

13. A method for facilitating reentry of a guidewire into a true lumen of a vessel, the method comprising:
advancing an elongate shaft with a capsule toward an occlusion in the vessel;
advancing the capsule through a subintimal space of the vessel and
positioning the capsule distal of the occlusion;
disposing an intravascular ultrasound (IVUS) catheter in the capsule;
re-entering a guidewire from the subintimal space into a true lumen of the vessel while imaging the guidewire with the IVUS catheter to ensure the guidewire is positioned and advanced correctly.

14. A system for facilitating reentry of a guidewire into a true lumen, the system comprising
an elongate shaft having a proximal end and a distal end;
a reentry guidewire lumen extending between the proximal end and a distal end, the reentry guidewire lumen configured to slidably receive a reentry guidewire;
an exit port fluidly connected to the reentry guidewire lumen at a distal end region of the elongate shaft;
an intravascular ultrasound (IVUS) lumen extending between the proximal end and the distal end of the elongate shaft, the IVUS lumen configured to slidably receive an IVUS catheter; and
an IVUS imaging window disposed in a sidewall of the elongate shaft in communication with the IVUS lumen and distal or adjacent to the exit port.

15. The system of claim 14, further comprising a guides in or adjacent to the exit port to deflect a reentry guidewire laterally relative to a long axis of the elongate shaft.

16. The system of claim 15, wherein the guide comprises: a ramp, a channel, or a deflector.

17. The system of claim 14, further comprising the reentry guidewire.

18. The system of claim 14, wherein the IVUS imaging window is positioned laterally adjacent to the exit port.

19. The system of claim 14, wherein the IVUS imaging window comprises a cut-out region.

20. The system of claim 14, further comprising one or more ultrasound markers on the distal end region of the elongate shaft.

21. The system of claim 14, further comprising one or more engagement features within the IVUS lumen configured to orient the IVUS catheter.

22. The system of claim 14, further comprising the IVUS catheter.

23. (canceled)
